(19) United States
(12) Patent Application Publication
(10) Pub. No.: US 2026/0063611 A1
(43) Pub. Date: Mar. 5, 2026

(54) SOIL GAS LOGGING SYSTEM

(71) Applicant: BOISE STATE UNIVERSITY, Boise, ID (US)

(72) Inventors: Jacob Anderson, Boise, ID (US); David Huber, Boise, ID (US); Owen Walsh, Boise, ID (US)

(73) Assignee: BOISE STATE UNIVERSITY, Boise, ID (US)

(21) Appl. No.: 19/318,214

(22) Filed: Sep. 3, 2025

Related U.S. Application Data
(60) Provisional application No. 63/690,910, filed on Sep. 5, 2024.

(51) Int. Cl. G01N 33/24; B33Y 10/00; B33Y 40/10; B33Y 80/00; G01N 33/00

(52) U.S. Cl. CPC G01N 33/24; B33Y 80/00; G01N 33/004; G01N 33/0047; B33Y 10/00; B33Y 40/10

(57) ABSTRACT

A gas logging system may include a data logger comprising an enclosure, a processor, and memory. The processor and the memory may be configured to log data associated with soil. The gas logging system may further include a set of probes. A probe of the set of probes may include a sensor enclosure, at least one gas sensor configured to provide the data associated with the soil to the processor and the memory, a gas-permeable water-impermeable membrane, a solid-state dehumidifying membrane, and a fan.

### CROSS-REFERENCE TO RELATED APPLICATIONS

This application claims the benefit of, U.S. Provisional Patent Application No. 63/690,910, filed Sep. 5, 2024, and entitled “Soil Gas Logging System,” the contents of which are incorporated by reference herein in their entirety.

### GOVERNMENT LICENSE RIGHTS

This invention was made with government support under 2331818, and 2331817 awarded by the National Science Foundation. The government has certain rights in the invention.

### FIELD OF THE DISCLOSURE

This disclosure is generally related to the field of soil testing and logging and, in particular, to a soil gas (e.g., CO₂, CH₄, O₂, etc.) logging system.

### BACKGROUND

Soil CO2 concentration and flux measurements are important in diverse fields, including geoscience, climate science, soil ecology, and agriculture. However, practitioners in these fields face difficulties with existing soil CO2 gas probes, which have had problems with high costs and frequent failures when deployed. The central challenge associated with soil gas probes is balancing the continuous exposure to soil moisture with keeping the sensor open to soil gases.

Three-dimensional (3D) printing may be an effective way to create a moisture barrier enclosure. However, current 3D printing methods and procedures may leave surfaces vulnerable to water permeability. An additional challenge is enabling gases to freely enter the enclosure while preventing water from doing the same. Further, gas entering the enclosure may include water vapor that should be mitigated within the enclosure. Other challenges and disadvantages may exist.

### SUMMARY

Disclosed is a gas logging system that resolves at least one of the challenges and disadvantages above. A 3D printed enclosure (which may be economical for small-scale production) may be formed following design principles that correct the usual water permeability flaw of 3D printed materials. Passive moisture protection measures include a hydrophobic, CO2-permeable PTFE membrane. Further, active moisture protection is conducted via a low-power micro-dehumidifier.

The disclosed gas logging system includes a data logger connected to several soil probes by cables. The data logger may sit on the ground surface (for easy user access) and the soil probes may be buried underground at depths of interest. Each probe may include a gas sensor along with a watertight enclosure and dehumidification system. The logger samples each soil probe at regular intervals and writes data to memory.

In an embodiment, a gas logging system includes a data logger including an enclosure, a processor, and memory. The processor and the memory are configured to log data associated with soil. The system further includes a set of probes, where a probe of the set of probes includes a sensor enclosure, at least one gas sensor configured to provide the data associated with the soil to the processor and the memory, a gas-permeable water-impermeable membrane, a solid-state dehumidifying membrane, and a fan.

In an embodiment, a gas logging method includes enclosing a processor and a memory in an enclosure of a data logger, the processor and memory configured to log data associated with soil. The method further includes enclosing one or more gas sensors in a sensor enclosure of a probe of a set of probes, where the gas sensor is configured to provide the data associated with the soil to the processor and memory. The method also includes activating a fan within the sensor enclosure to create airflow at a gas-permeable water-impermeable membrane. The method includes activating a solid-state dehumidifying membrane within the sensor enclosure.

In an embodiment, a method includes forming a sensor enclosure using a printer filament in an additive manufacturing process. The method further includes positioning a fan within the sensor enclosure. The method also includes attaching a gas-permeable water-impermeable membrane to the enclosure. The method includes attaching a solid-state dehumidifying membrane to the enclosure. The method further includes enclosing a gas sensor within the sensor enclosure.

In some embodiments, the method includes drying the printer filament, where the printer filament comprises acrylonitrile styrene acrylate (ASA), where the sensor enclosure includes a wall that is at least 1.0 mm thick, where the sensor enclosure has randomized seams between layers, where forming the sensor enclosure body is performed using a k-value that is equal to or greater than 0.98, and where the method further includes treating a surface of the enclosure body with acetone.

### BRIEF DESCRIPTION OF THE DRAWINGS

FIG. 1 is a block diagram of an embodiment of a gas logging system.

FIG. 2 is an exploded view of an embodiment of a sensor enclosure.

FIG. 3 is an exploded view of an embodiment of a sensor enclosure.

FIG. 4 is a set of graphs depicting gas logging data.

FIG. 5 is a flow chart depicting a gas logging method.

FIG. 6 is a flow chart depicting a gas logger probe construction method.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the scope of the disclosure.

### DETAILED DESCRIPTION

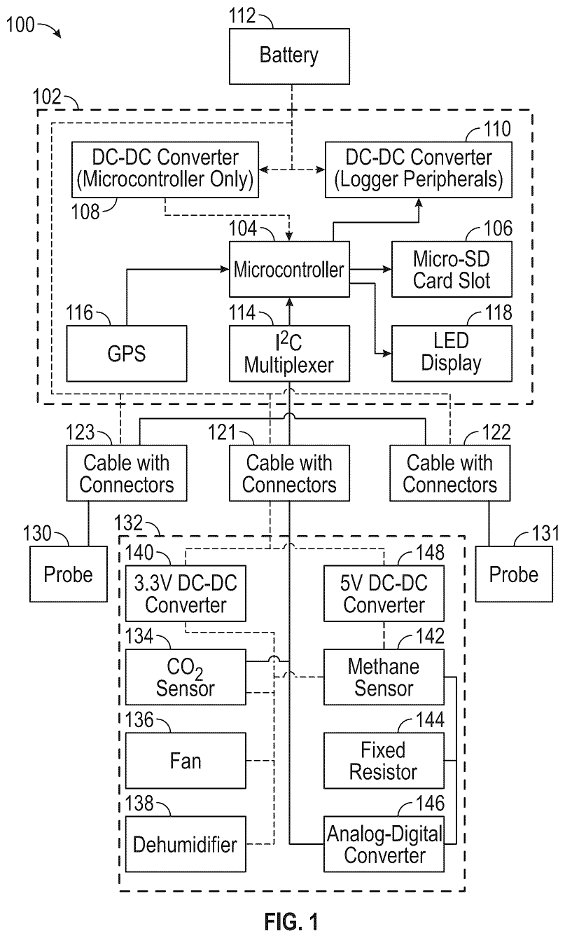

Referring to FIG. 1, a gas logging system 100 is depicted. The system 100 may include a data logger 102 comprising an enclosure (represented by the upper dotted box shown in FIG. 1), a processor 104, and memory 106. The processor 104 and the memory 106 may be configured to log data associated with soil as described herein.

The processor 104 may include a central processing unit (CPU), a graphical processing unit (GPU), a digital signal processor (DSP), a peripheral interface controller (PIC), another type of microprocessor or microcontroller, and/or combinations thereof. Further, the processor 104 may be implemented as an integrated circuit, field-programmable gate array (FPGA), application-specific integrated circuit (ASIC), combination of logic gate circuitry, another type of digital or analog electrical design components, or combinations thereof. The memory 106 may include memory devices such as random-access memory (RAM), read-only memory (ROM), magnetic disk memory, optical disk memory, flash memory, another type of memory capable of storing data and processor instructions, or the like, or combinations thereof. In an embodiment, and as shown in FIG. 1, the memory includes a microSD card.

To power the processor 108, the data logger 102 may include a first direct-current-direct-current (DC-DC) converter 108. To power peripherals, including probes, the data logger 102 may include a second DC-DC converter 110. In some embodiments, a single DC-DC converter may provide power to the entire system. An external power source 112, such as a batter, may power each of the DC-DC converters 108, 110. Many battery types may be compatible with the system 100. In an embodiment, the external power source 112 may include a 12-volt lead-acid battery.

The data logger 102 may further include a global positioning system (GPS) unit 116, a light-emitting-diode (LED) display 118, and a multiplexor 114, which may be powered by the second DC-DC converter 110. The GPS unit 116 may be used by the processor 104 as a source for date and time information. The multiplexor 114 may provide the data associated with the soil from a set of probes 130, 131, 132 to the processor 104 and the memory 106. In an embodiment, the multiplexor 114 is a I2C multiplexer (over I2C). The set of probes 130, 131, 132 may receive power and communicate with the data logger 102 via a set of cables 121, 122, 123. Each of the set of cables 121, 122, 123 may be up to 25 meters in length and may connect to bulkhead connectors on both a respective probe and data logger. Although FIG. 1 depicts three probes with three cables, any number of probes and cables may be used with the system 100. In an embodiment, the multiplexer 114 is connected to up to eight bulkhead connectors mounted on the outside of the data logger enclosure. Further, although not shown in FIG. 1, in some embodiments, the data logging system 100 may connect to a laptop with a USB-C cable in order to view real-time output and update firmware.

For clarity, one probe 132 of the set of probes 130, 131, 132 is described with reference to FIG. 1. It should be understood that each of the set of probes 130, 131, 132 may be similarly described. The probe 132 may include a sensor enclosure (represented by the lower dotted box shown in FIG. 1). The sensor enclosure may be configured with a gas-permeable water-impermeable membrane as described further herein. The probe 132 may further include at least one gas sensor. For example, the probe 132 may include a carbon-dioxide sensor 134, a methane sensor 142 or both. The carbon-dioxide sensor 134 may be a photoacoustic carbon-dioxide sensor, and in particular, an SCD41 photoacoustic non-dispersive infrared (NDIR) carbon-dioxide sensor. The methane sensor 142 may be An MQ-4 electrochemical methane sensor. The at least one gas sensor (e.g., the carbon dioxide sensor 134 and/or the methane sensor 142) may be configured to provide the data associated with the soil to the processor 104 and the memory 106. Each of the probes 130, 131, 132 may be powered by the second DC-DC converter 110.

In embodiments where the probe 132 includes the methane sensor 142, the probe 132 may also include a fixed resistor 144, an analog-digital converter 146, and a 5-volt DC-DC converter 148. The fixed resistor 144 may form a bridge circuit that can be configured with the methane sensor 142 to sense methane gas. For example, the analog-digital converter 146 can measure a voltage across the fixed resistor 144 as an indicator of methane concentration. In post-processing, sensor resistance may be calculated from the measured voltage, and a methane-to-resistance relationship taken from known values listed in a datasheet or an independent calibration may be used to infer methane concentration. Because temperature and humidity may have secondary effects on the methane sensor's reading, it may be advisable to measure these as well. In some embodiments, the carbon-dioxide sensor may have a built-in thermometer and humidity sensors and can provide this information to the processor 104 and the memory 106.

The probe 132 may include a fan 136, a solid-state dehumidifying membrane 138, and a 3.3 volt DC-DC converter 140. The solid-state dehumidifying membrane may include a solid polymer electrolyte member. In response to a direct current applied to the solid polymer electrolyte member, hydrogen ions at an anode of the solid polymer electrolyte member may be separated from water molecules in water vapor and may be transported to a cathode side of the solid polymer electrolyte member and discharge from the sensor enclosure. The 3.3 volt DC-DC converter 140 may be configured to power the at least one gas sensor (e.g., the carbon dioxide sensor 134 and/or the methane sensor 142), the solid-state dehumidifying membrane 138, and the fan 136.

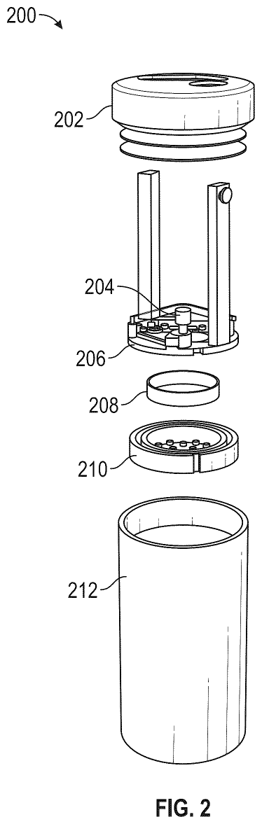

Referring to FIG. 2, an embodiment of a sensor enclosure 200 is depicted. The sensor enclosure 200 may include a cap 202, with openings for an active membrane (e.g., the solid-state dehumidifying membrane 138) and a cable (sealed with O-rings), a sensor circuit board 204 for holding the at least one gas sensor (e.g., the carbon-dioxide sensor 134, the methane sensor 142, or both), a rack 206 with lock pins for mounting the sensor circuit board, a ring for attaching a gas-permeable water-impermeable membrane 208, a membrane screen 210 (sealed with O-rings above and below), and a probe housing 112.

The enclosure 200 may include a bulkhead connector mounted on the cap 202 as described herein to connect to a cable (e.g., the cables 121, 122, 123). Humidity (the primary environmental challenge in the often-wet soil setting) may be managed by a combination of the enclosure's watertightness, the use of the gas-permeable water-impermeable membrane 208 for gas exchange with the soil, and the fan 136 (shown in FIG. 1) circulating air continuously inside the enclosure 200, and a solid-state dehumidifying membrane 138 (also shown in FIG. 1) for removing water vapor from inside the enclosure.

In some embodiments, the gas-permeable water-impermeable membrane 208 may include polytetrafluoroethylene (PTFE). The body 212 of the enclosure 200 may be a water-resistant body, and may be formed from acrylonitrile styrene acrylate (ASA). The body 212 of the sensor enclosure 200 may have a shell thickness of at least 1.0 mm.

To form the enclosure 200, printer filament, including ASA, may be dried and the sensor enclosure 200 may be formed with randomized seams between layers. Forming the sensor enclosure 200 may be performed using a k-value that is equal to or greater than 0.98. A surface of the enclosure 200 may further be treated with acetone. In this way, the enclosure 200 may be less water permeable than a typical 3D printed enclosure.

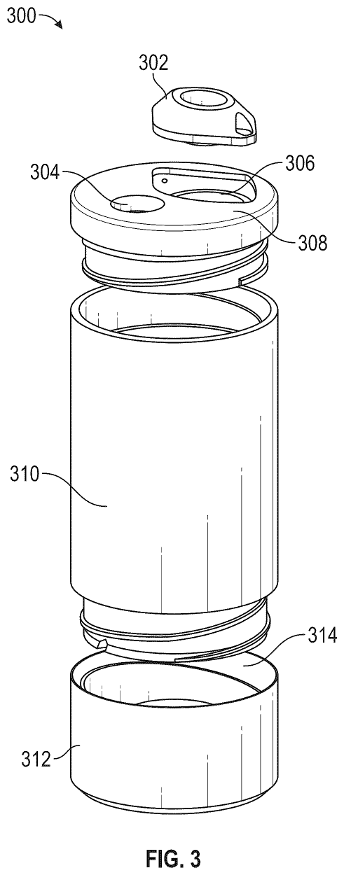

Referring to FIG. 3, an embodiment of a sensor enclosure 300 is depicted. The enclosure 300 may include a first part 302 of a cap and a second part 304 of the cap. Within the second part 304 of the cap, a first gas-permeable water-impermeable membrane 306 and a solid-state dehumidifying membrane 308 may be attached. The enclosure 300 may further include a body 310 and a removable lower portion 312. Within the removable lower portion 312, a second gas-permeable water-impermeable membrane 314 may be attached.

The enclosure 300 may be similar to the enclosure 200 in most ways, with the exception of the removable bottom portion 312. In this embodiment, the first gas-permeable water-impermeable membrane 306 and the second gas-permeable water-impermeable membrane 314 cover openings at the top and at the bottom of the body 310 by being positioned outside the opening near the fan where most gas exchange occurs, and outside the dehumidifier membrane (e.g., the solid-state dehumidifying membrane 308) where water vapor is removed.

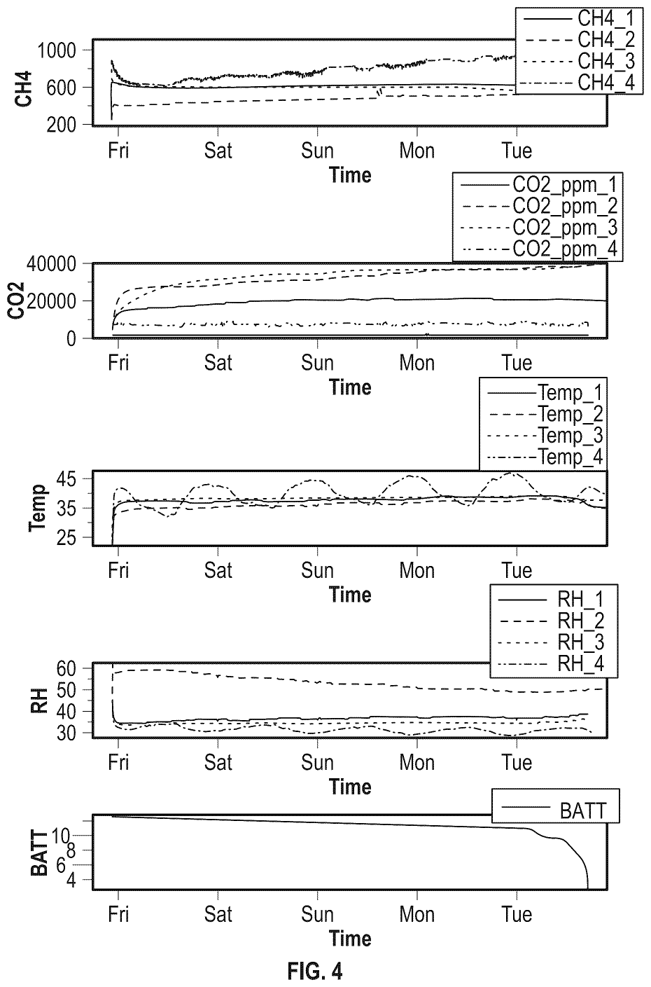

Referring to FIG. 4, a set of graphs depicting gas logging data are provided. Each graph depicts four channels, corresponding respectively to four probes positioned at different locations and/or depths. The first graph (from the top) shows measurements of methane (CH4). The second graph shows measurements of carbon-dioxide (CO2). The third graph shows measurements of temperature. The fourth graph shows measurements of humidity. The fifth graph shows a voltage of the battery used to power the system. As a note, the fourth sensor in each key was placed at the surface and had much lower carbon-dioxide than the other probes, so its carbon dioxide readings are multiplied by a factor of 10 for visibility.

As demonstrated by FIG. 4, the system 100 may successfully measure levels of carbon-dioxide gas, methane gas, temperature, and humidity at different levels and locations within soil. By mitigating moisture within the probes, these measurements may be taken over longer periods of time and the system 100 may be more robust than typical soil measurement systems.

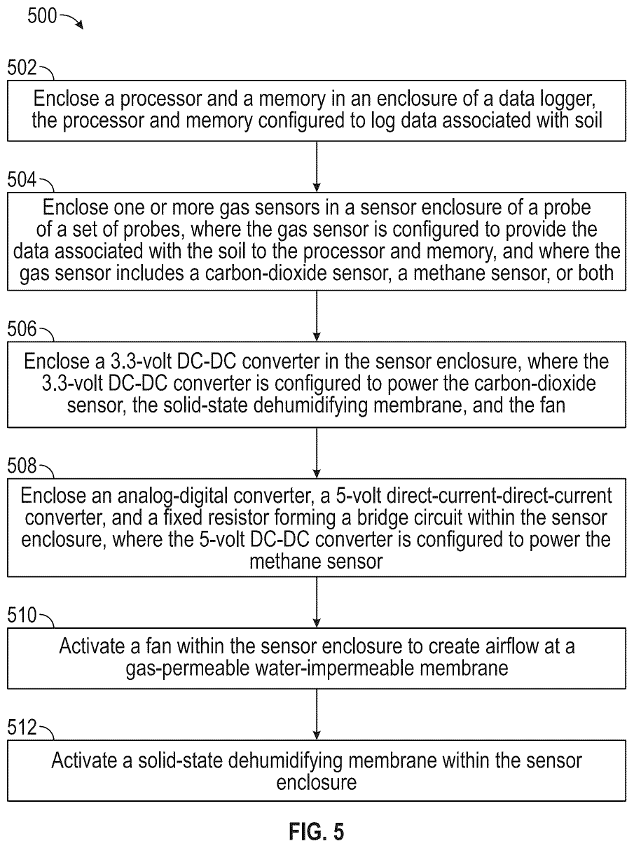

Referring to FIG. 5, a gas logging method 500 is depicted. The method 500 may include enclosing a processor and a memory in an enclosure of a data logger, the processor and memory configured to log data associated with soil, at 502. For example, the processor 104 and the memory 106 may be enclosed in the enclosure of the data logger 102.

The method 500 may further include enclosing one or more gas sensors in a sensor enclosure of a probe of a set of probes, where the gas sensor is configured to provide the data associated with the soil to the processor and memory, and where the gas sensor includes a carbon-dioxide sensor, a methane sensor, or both, at 504. For example, the carbon-dioxide sensor 134 and/or the methane sensor 142 may be enclosed in the sensor enclosure 200 or the sensor enclosure 300.

The method 500 may also include enclosing a 3.3-volt DC-DC converter in the sensor enclosure, where the 3.3-volt DC-DC converter is configured to power the carbon-dioxide sensor, the solid-state dehumidifying membrane, and the fan, at 506. For example, the 3.3-volt DC-DC converter 140 may be enclosed int the sensor enclosure 200 or the sensor enclosure 300.

The method 500 may include, enclosing an analog-digital converter, a 5-volt direct-current-direct-current converter, and a fixed resistor forming a bridge circuit within the sensor enclosure, where the 5-volt DC-DC converter is configured to power the methane sensor, at 508. For example, the analog-digital converter 146, the 5-volt DC-DC converter 148, and the fixed resistor 144 may be enclosed within the sensor enclosure 200 or the sensor enclosure 300.

The method 500 may also include activating a fan within the sensor enclosure to create airflow at a gas-permeable water-impermeable membrane, at 510. For example, the fan 136 may be activated to generate airflow.

The method 500 may include activating a solid-state dehumidifying membrane within the sensor enclosure, at 512. For example, the solid-state dehumidifying membrane 138 may be activated.

A benefit of the method 500 is that gas logging may be performed while actively mitigating moisture accumulation within a probe enclosure. Other benefits or advantages may exist.

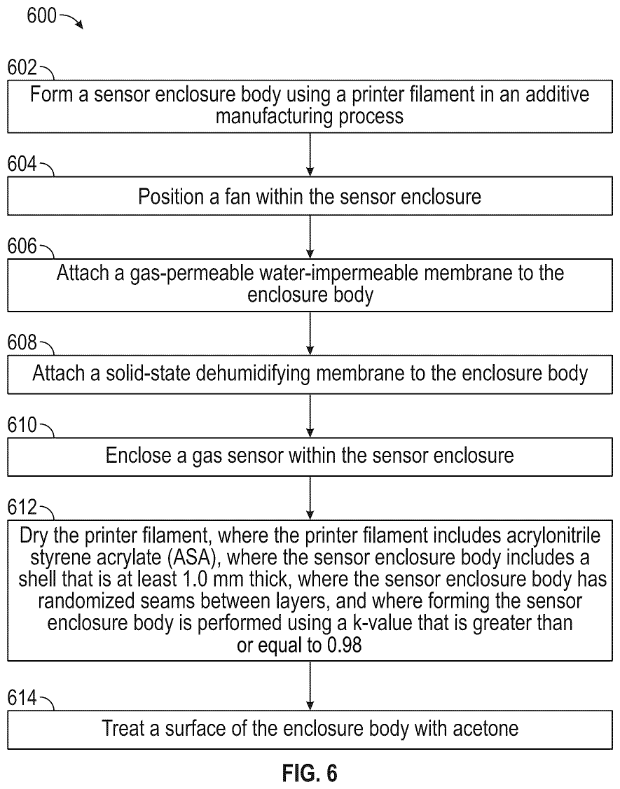

Referring to FIG. 6, a gas logger probe construction method 600 is depicted. The method 600 may include forming a sensor enclosure body using a printer filament in an additive manufacturing process, at 602. For example, the sensor enclosure 200 or the sensor enclosure 300 may be printed using an additive manufacturing process.

The method 600 may further include positioning a fan within the sensor enclosure, at 604. For example, the fan 136 may be positioned within the sensor enclosure 200 or the sensor enclosure 300.

The method 600 may also include attaching a gas-permeable water-impermeable membrane to the enclosure body, at 606. For example, the gas-permeable water-impermeable membrane 208 may be attached to the sensor enclosure 200 or as described with respect to the sensor enclosure 300, multiple gas-permeable water-impermeable membranes may be attached to each opening of the sensor enclosure 300.

The method 600 may include attaching a solid-state dehumidifying membrane to the enclosure body, at 608. For example, the solid-state dehumidifying membrane 138 may be attached within the sensor enclosure 200 or the sensor enclosure 300.

The method 600 may further include enclosing a gas sensor within the sensor enclosure, at 610. For example, the carbon-dioxide sensor 134, the methane sensor 142, or both, may be enclosed with the sensor enclosure 200 or the sensor enclosure 300.

The method 600 may also include drying the printer filament, where the printer filament comprises acrylonitrile styrene acrylate (ASA), where the sensor enclosure body includes a shell that is at least 1.0 mm thick, where the sensor enclosure body has randomized seams between layers, and where forming the sensor enclosure body is performed using a k-value that is equal to or greater than 0.98, at 612.

The method 600 may include treating a surface of the enclosure body with acetone, at 614.

The method 600 may overcome challenges with 3D printing a water impermeable container. Other benefits or advantages may exist.

Although various embodiments have been shown and described, the present disclosure is not so limited and will be understood to include all such modifications and variations as would be apparent to one skilled in the art.

What is claimed is:

1. A gas logging system comprising:
a data logger comprising an enclosure, a processor, and memory, wherein the processor and the memory are configured to log data associated with soil; and
a set of probes, wherein a probe of the set of probes comprises a sensor enclosure, at least one gas sensor configured to provide the data associated with the soil to the processor and the memory, a gas-permeable water-impermeable membrane, a solid-state dehumidifying membrane, and a fan.

2. The system of claim 1, wherein the probe further comprises a 3.3 volt direct-current-direct-current converter configured to power the at least one gas sensor, the solid-state dehumidifying membrane, and the fan.

3. The system of claim 1, wherein the at least one gas sensor is a carbon-dioxide sensor.

4. The system of claim 1, wherein the at least one gas sensor is a methane sensor.

5. The system of claim 4, wherein the probe further comprises an analog-digital converter, a 5-volt direct-current-direct-current converter, and a fixed resistor forming a bridge circuit, configured with the methane sensor to sense methane.

6. The system of claim 1, wherein the data logger further comprises one or more direct-current-direct current converters, a global positioning system unit, a light-emitting-diode display, and a multiplexor that provides the data associated with the soil from the set of probes to the processor and the memory.

7. The system of claim 1, wherein the gas-permeable water-impermeable membrane comprises polytetrafluoroethylene (PTFE).

8. The system of claim 1, wherein the solid-state dehumidifying membrane includes a solid polymer electrolyte member, wherein in response to a direct current applied to the solid polymer electrolyte member, hydrogen ions at an anode of the solid polymer electrolyte member are separated from H₂O molecules in water vapor and transported to a cathode side of the solid polymer electrolyte member and discharge from the sensor enclosure.

9. The system of claim 1, wherein the sensor enclosure comprises a water-resistant body.

10. The system of claim 9, wherein the water-resistant body comprises acrylonitrile styrene acrylate (ASA).

11. The system of claim 10, wherein the water-resistant body of the sensor enclosure further comprises at least 1.0 mm ASA shell thickness.

12. The system of claim 1, further comprising one or more cables with connectors placing the at least one sensor in communication with the processor.

13. A gas logging method comprising:
enclosing a processor and a memory in an enclosure of a data logger, the processor and memory configured to log data associated with soil; and
enclosing one or more gas sensors in a sensor enclosure of a probe of a set of probes, wherein the gas sensor is configured to provide the data associated with the soil to the processor and memory;
activating a fan within the sensor enclosure to create airflow at a gas-permeable water-impermeable membrane; and
activating a solid-state dehumidifying membrane within the sensor enclosure.

14. The method of claim 13, further comprising enclosing a 3.3-volt direct-current-direct-current converter in the sensor enclosure, wherein the 3.3-volt direct-current-direct-current converter is configured to power the gas sensor, the solid-state dehumidifying membrane, and the fan.

15. The method of claim 13, wherein the at least one gas sensor includes a carbon-dioxide sensor, a methane sensor, or both.

16. The method of claim 13, wherein the at least one gas sensor is a methane sensor, and wherein the method further comprises enclosing an analog-digital converter, a 5-volt direct-current-direct-current converter, and a fixed resistor forming a bridge circuit within the sensor enclosure.

17. The method of claim 13, wherein the solid-state dehumidifying membrane includes a solid polymer electrolyte member, wherein in response to a direct current applied to the solid polymer electrolyte member, hydrogen ions at an anode of the solid polymer electrolyte member are separated from H₂O molecules in water vapor and transported to a cathode side of the solid polymer electrolyte member and discharge from the sensor enclosure.

18. A method comprising:
forming a sensor enclosure body using a printer filament in an additive manufacturing process;
positioning a fan within the sensor enclosure;
attaching a gas-permeable water-impermeable membrane to the enclosure body;
attaching a solid-state dehumidifying membrane to the enclosure body; and
enclosing a gas sensor within the sensor enclosure.

19. The method of claim 18, wherein the method further comprises drying the printer filament, wherein the printer filament comprises acrylonitrile styrene acrylate (ASA), wherein the sensor enclosure body includes a shell that is at least 1.0 mm thick, wherein the sensor enclosure body has randomized seams between layers, wherein forming the sensor enclosure body is performed using a k-value that is equal to or greater than 0.98, and wherein the method further comprises treating a surface of the enclosure body with acetone.

20. The method of claim 18, further comprising configuring the gas sensor to provide data associated with soil to a processor and memory enclosed in a data logger enclosure.
